(19) United States
(12) Patent Application Publication
(10) Pub. No.: US 2009/0030208 A1
(43) Pub. Date: Jan. 29, 2009

(54) (+) - and (-) -8-Alkyl-3-(Trifluoralkylsulfonyloxy)-8-Azabicyclo(3.2.1.)Oct-2-Ene

(76) Inventors: Dan Peters, Malmo (SE); Dorthe Filtenborg Olesen, Ballerup (DK); Eva Dam, Roskilde (DK)

Correspondence Address: BIRCH STEWART KOLASCH & BIRCH, PO BOX 747, FALLS CHURCH, VA, 22040-0747, US

(21) Appl. No.: 11/886,669

(22) PCT Filed: Apr. 6, 2006
(86) PCT No.: PCT/EP2006/061364

§ 371 Date: Sep. 19, 2007

Related U.S. Application Data
(60) Provisional application No. 60/669,917, filed on Apr. 11, 2005.

(30) Foreign Application Priority Data
Apr. 8, 2005 (DK) PA 2005 00514

(51) Int. Cl. C07D 221/22

(52) U.S. Cl. 546/183

(57) ABSTRACT

This invention relates to novel enantiopure compounds, useful as starting material for synthesis of enantiopure pharmaceuticals.

In other aspects the invention relates to a method of preparing the enantiopure compounds of the invention.

### TECHNICAL FIELD

This invention relates to novel enantiopure compounds, useful as starting material for synthesis of enantiopure pharmaceuticals.

In other aspects the invention relates to a method of preparing the enantiopure compounds of the invention.

### BACKGROUND ART

Tropinone (8-methyl-8-azabicyclo[3.2.1]octan-3-one) is a useful starting material for the synthesis of many pharmaceutical compounds—cf. e.g. WO 97/13770, Example 1 (NeuroSearch A/S). However, using tropinone as a starting material in an achiral synthesis will in some cases result in products being racemates of two enantiomers.

Often it is desirable to synthesise enantiopure compounds rather than the racemate as the two enantiomers may have different pharmacological profiles.

Further it is often desirable, and sometimes subject to regulatory demands, to undertake drug development on specific enantiomers rather than racemic drugs. This rationale is based on the findings that often the desired characteristics of chiral compounds reside with one of its enantiomers, while the other enantiomer might in fact add to a potential toxicological effect of the drug.

Also, in order to allow thorough investigation of each enantiomer, enantiopure compounds and processes for obtaining enantiopure such compounds of chiral compounds are of significant importance for drug development.

### SUMMARY OF THE INVENTION

In its first aspect, the invention provides an enantiopure compound of the Formula I

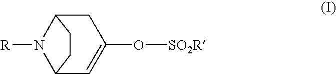

or an addition salt thereof; wherein R and R′ are as defined below.

In its second aspect, the invention provides a method of preparing the enantiopure compound.

Other objects of the invention will be apparent to the person skilled in the art from the following detailed description and examples.

### DETAILED DISCLOSURE OF THE INVENTION

### Enantiopure Compounds

In its first aspect the present invention provides an enantiopure compound of the Formula I

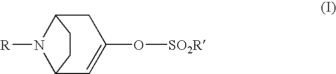

or an addition salt thereof;
wherein
R represents alkyl or a protection group; and
R′ represents perfluoroalkyl.

In one embodiment, R represents alkyl. In a special embodiment, R represents methyl.

In a further embodiment, R represents benzyl, BOC (t-butoxycarbonyl), Fmoc (9-fluorenylmethoxycarbonyl) or any other suitable protection group.

In a still further embodiment, R′ represents trifluoromethyl.

In a still further embodiment, the present invention provides enantiopure 8-methyl-3-(trifluoromethylsulfonyloxy)-8-azabicyclo[3.2.1]oct-2-ene or an addition salt thereof.

In a further embodiment, the chemical compound of the invention is enantiopure (+)-8-methyl-3-(trifluoromethylsulfonyloxy)-8-azabicyclo[3.2.1]oct-2-ene or an addition salt thereof.

In a still further embodiment, the chemical compound of the invention is enantiopure (−)-8-methyl-3-(trifluoromethylsulfonyloxy)-8-azabicyclo[3.2.1]oct-2-ene or an addition salt thereof.

### Methods of Preparation

In its first aspect the present invention provides a method for preparing an enantiopure compound of the Formula I

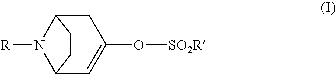

or an addition salt thereof; wherein
R represents alkyl or a protection group;
R′ represents perfluoroalkyl;
which method comprises reacting a compound of formula II

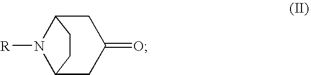

with the relevant N-phenyl-bis(perfluoroalkylsulphon)imide or a functional equivalent thereof in the presence of a chiral lithium amide.

In one embodiment, R represents alkyl. In a special embodiment, R represents methyl.

In a further embodiment, R represents benzyl, BOC (t-butoxycarbonyl), Fmoc (9-fluorenylmethoxycarbonyl) or any other suitable protection group.

In a still further embodiment, R′ is trifluoromethyl or nonafluorobutyl.

In a still further embodiment, the N-phenyl-bis(perfluoroalkylsulphon)imide or a functional equivalent is selected from the group of N-phenyl-bis(trifluoromethanesulphon)imide, trifluoromethanesulfonic anhydride, trifluoro-methanesulfonyl chloride, N-(5-chloro-2-pyridyl)bis(trifluoromethanesulfon)imide, N-(2-pyridyl)bis(trifluoromethanesulfon)imide and trifluoro-methanesulfonic acid methyl ester.

In a further embodiment, the chiral lithium amide is a lithium methylbenzylamide. In a special embodiment, the chiral lithium amide is N-lithium bis-α-methylbenzylamide.

In a special embodiment, the chiral lithium amide for the reaction is formed by reaction between a chiral amine and a lithiating agent.

In a further special embodiment, the chiral amine is (+)-bis-α-methyl-benzylamine or (−)-bis-α-methyl-benzylamine and the lithiating agent is butyllithium.

In one embodiment, the method for preparing the enantiopure compound of the Formula I may be performed as a one-pot synthesis.

In a further embodiment, the method for preparing the enantiopure compound of the Formula I may be performed by the steps:

(1) adding the compound of formula II to a mixture containing the chiral lithium amide; followed by
(2) adding the relevant N-phenyl-bis(perfluoroalkylsulphon)imide or a functional equivalent thereof to the mixture of step (1).

In a still further embodiment, the above step (1) is performed by the step:

(1a) mixing the chiral amine with the lithiating agent; followed by
(1b) adding the compound of formula II to the mixture of step (1).

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

Any combination of two or more of the embodiments as described above is considered within the scope of the present invention.

### Definition of Substituents

In the context of this invention an alkyl group designates a univalent saturated, straight or branched hydrocarbon chain. The hydrocarbon chain preferably contains of from one to eight carbon atoms (C₁₋₈-alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a perfluoroalkyl group designates an alkyl group having all hydrogen atoms replaced with fluoro atoms. Examples include trifluoromethyl, pentafluoroethyl, heptafluoropropyl and nonafluorobutyl.

### Enantiopurity

In the context of this invention a compound being enantiopure means that the compound is in enantiomeric excess of at least 80% (w/w) over the opposite enantiomer. In one embodiment, the enantiopure compound is in enantiomeric excess of at least 85%, 88% or 90% over the opposite enantiomer. In a further embodiment, the enantiopure compound is in enantiomeric excess of at least 95%, 98%, or 99% over the opposite enantiomer.

### Chiral Amine

Chiral amines are useful—in the form of the equivalent lithium amide—for the asymmetric transformation of ketones. Such chiral amines are well known and described in the art. These amines include, for example, (+)- and (−)-bis-α-methyl-benzylamine.

### Protection Groups

Protection of amino groups against reaction during one or more synthesis steps is a procedure well known and described in the art. Examples of suitable protection groups are those which are customarily used in peptide synthesis. Specific examples include, e.g., benzyl, BOC (t-butoxycarbonyl), Fmoc (9-fluorenylmethoxycarbonyl) or any other suitable protection group. Further details on suitable protection groups may be found in “Protective groups in organic synthesis”, Greene T W and Wits P G (John Wiley & Sons, Inc. New York, 1999).

### Addition Salts

The chemical compound of the invention may be provided in any form suitable as a starting material for further synthesis. Suitable forms include addition salts.

Examples of addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be also useful.

In the context of this invention the “onium salts” of N-containing compounds are also contemplated as acceptable addition salts. Preferred “onium salts” include the alkyl-onium salts, the cycloalkyl-onium salts, and the cycloalkylalkyl-onium salts.

### EXAMPLES

The invention is further illustrated with reference to the following examples, which are not intended to be in any way limiting to the scope of the invention as claimed.

### (−)-8-Methyl-3-(trifluoromethylsulfonyloxy)-8-azabicyclo[3.2.1]oct-2-ene

To a stirred mixture of [S—(R*, R*)](−)-bis-α-methyl-benzylamine hydrochloric acid salt ([α]_{D}²⁵=(−)-73.2°) (86.5 g, 0.33 mmol) and tetrahydrofuran (1000 ml) was added at <5° C.: Butyllithium (264 ml, 2.5 M). The mixture was stirred at 0° C. for 1 h. The mixture was cooled to −70° C. and tropinone (41.8 g, 0.3 mmol) solved in tetrahydrofuran (200 ml) was added over a period of 90 min. The mixture was stirred for 3 h at −70° C. N-phenyl-bis(trifluoromethanesulfon)imide (114.3 g, 0.32 mmol) solved in tetrahydrofuran was added to the mixture <70° C. over 2 h time period. The mixture was allowed to reach room temperature over night. Water (3 L) was added followed by extraction with diethylether (2×1 L). The organic phase was washed with water (2×1 L). The crude mixture of the title product and the chiral amine was separated by silica gel (1 kg) column chromatography using ethyl acetate initially in order to eluate the chiral amine and then use a mixture of methanol and dichloromethane (2:8). The product was isolated in 78% (0.233 mol).

The stereochemistry of the product was confirmed by derivatisation to (−)-3-(2-benzothienyl)-8-methyl-8-azabicyclo[3.2.1]oct-2-ene:

A mixture of (−)-8-methyl-3-(trifluoromethylsulfonyloxy)-8-azabicyclo[3.2.1]oct-2-ene, (4.64 g, 17.1 mmol), 1,2-dimethoxyethane (100 ml), 2-benzothienyl boronic acid (4.5 g, 25 mmol), potassium carbonate (9.2 g, 66.6 mmol), lithium chloride (2.0 g, 47.2 mmol) and water (50 ml) was bubbled through with argon for 10 min. Pd(PPh3)₄ (0.17 g, 0.13 mmol) was added followed by reflux for 45 min. The mixture was allowed to cool to room temperature. Water (100 ml) was added followed by extraction with diethyl ether (2×50 ml). The organic phase was washed with water (2×50 ml). The organic phase was dried and evaporated. The hydrochloric acid salt was precipitated by addition of hydrochloric acid (4 M) solved in ethanol (5 ml, 96%). Addition water (50 ml) and concentrated ammonia (50 ml) followed by extraction with dichloromethane (2×50 ml) gave the free base. (4.09 g, 100% from the salt) [α]_{D}²⁵=(−)-46.3°. The tartaric acid salt was prepared by adding D-tartaric acid (2.4 g, 16 mmol) to a mixture of the free base and ethanol (96%) at reflux. The mixture was allowed to cool overnight and was isolated by filtration. Yield 5.06 g (12.47 mmol), chiral HPLC (−) 94.9% and (+) 5.1%. Recrystallization of 4.85 g (11.9 mmol) from ethanol (150 ml, 96%) yielded (3.26 g, 8.0 mmol), chiral HPLC (−) 97.9% and (+) 2.1%. Mp 67.6-76.0° C.

[The chiral purities of the products were analyzed by the following HPLC method: Column: ChromTech Chiral-AGP, 100×4.6 mm, 5 μm. Temperature: 25° C. Flow: 0.9 ml/min. Injection volume: 10 μl. Detection: UV 290 nm. Mobile phase: 5 mM Sodium acetate buffer pH 5.0 containing 3% v/v acetonitrile.]

### (+)-8-Methyl-3-(trifluoromethylsulfonyloxy)-8-azabicyclo[3.2.1]oct-2-ene

Was prepared according to method C using the other chiral amine [R—(R*, R*)](+)-bis-α-methyl-benzylamine hydrochloric acid salt, [α]_{D}²⁵=(+)-73.8°.

The stereochemistry of the product was confirmed by derivatisation to (+)-3-(2-benzothienyl)-8-methyl-8-azabicyclo[3.2.1]oct-2-ene as described above: After first recrystallization converted to free base, [α]_{D}²⁵=(+)-46.3°. After second recrystallization converted to free base, [α]_{D}²⁵=(+)-53.3°. Chiral HPLC (+) 98.3% and (−) 1.7%. Mp 66.2-73.6° C.

1. An enantiopure compound of Formula I 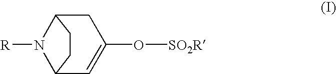
or an addition salt thereof;
wherein
R represents alkyl or a protection group; and
R′ represents perfluoroalkyl.

2. The chemical compound of claim 1, or an addition salt thereof, wherein
R represents alkyl.

3. The chemical compound of claim 1, or an addition salt thereof, wherein
R′ represents trifluoromethyl.

4. The chemical compound of claim 1, being
enantiopure (+)-8-Methyl-3-(trifluoromethylsulfonyloxy)-8-azabicyclo[3.2.1]oct-2-ene; or an addition salt thereof.

5. The chemical compound of claim 1, being
enantiopure (−)-8-Methyl-3-(trifluoromethylsulfonyloxy)-8-azabicyclo[3.2.1]oct-2-ene; or an addition salt thereof.

6. A method for preparing an enantiopure compound of Formula I 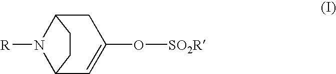
or an addition salt thereof;
wherein
R represents alkyl or a protection group; and
R′ represents perfluoroalkyl;
which method comprises reacting a compound of formula II 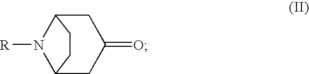
with the relevant N-phenyl-bis(perfluoroalkylsulphon)imide or a functional equivalent thereof in the presence of a chiral lithium amide.

7. The method of claim 6, wherein R is alkyl.

8. The method of claim 6, wherein R′ is trifluoromethyl.

9. The method of claim 6, wherein the N-phenyl-bis(perfluoroalkylsulphon)imide or a functional equivalent is selected from the group of N-phenyl-bis(trifluoromethanesulphon)imide, trifluoromethanesulfonic anhydride, trifluoro-methanesulfonyl chloride, N-(5-chloro-2-pyridyl)bis(trifluoromethanesulfon)imide, N-(2-pyridyl)bis(trifluoromethanesulfon)imide and trifluoromethanesulfonic acid methyl ester.

10. The method of claim 6, wherein the chiral lithium amide is a lithium methylbenzylamide.
